(19) United States
(12) Patent Application Publication
(10) Pub. No.: US 2011/0105574 A1
(43) Pub. Date: May 5, 2011

(54) PAI-1 EXPRESSION AND ACTIVITY INHIBITORS FOR THE TREATMENT OF OCULAR DISORDERS

(75) Inventors: Debra L. Fleenor, Crowley, TX (US); Allan R. Shepard, Fort Worth, TX (US); Iok-Hou Pang, Grand Prairie, TX (US); Mark R. Hellberg, Arlington, TX (US); Abbot F. Clark, Arlington, TX (US)

(73) Assignee: ALCON RESEARCH, LTD., Fort Worth, TX (US)

(21) Appl. No.: 13/006,984

(22) Filed: Jan. 14, 2011

Related U.S. Application Data
(63) Continuation of application No. 12/421,456, filed on Apr. 9, 2009.
Application No. 12/421,456 is a continuation-in-part of application No. 11/931,393, filed on Oct. 31, 2007, now abandoned.
(60) Provisional application No. 61/048,176, filed on Apr. 26, 2008.
(60) Provisional application No. 60/863,715, filed on Oct. 31, 2006.

(51) Int. Cl. A61K 31/41; A61K 31/403; A61P 27/02

(52) U.S. Cl. 514/381; 514/419

(57) ABSTRACT

The invention concerns in one embodiment a method for treating glaucoma or elevated IOP in a patient comprising administering to the patient an effective amount of a composition comprising an agent that inhibits PAI-1 expression or PAI-1 activity. Another embodiment of the present invention is a method of treating a PAI-1-associated ocular disorder in a subject in need, comprising administering to the patient an effective amount of a composition comprising an agent that inhibits PAI-1 activity or expression.

### CROSS-REFERENCE TO RELATED APPLICATIONS

This application is a continuation of U.S. application Ser. No. 12/421,456 filed Apr. 9, 2009, which is a continuation-in-part of U.S. application Ser. No. 11/931,393 filed Oct. 31, 2007, now abandoned, priority of which is claimed under 35 U.S.C. §120, the contents of which are hereby incorporated by reference. The present application also claims priority under 35 U.S.C. §119 to U.S. Provisional Patent Application Ser. No. 61/048,176 filed Apr. 26, 2008, and U.S. Provisional Patent Application Ser. No. 60/863,715 filed Oct. 31, 2006 the entire contents of which are incorporated herein by reference.

### TECHNICAL FIELD OF THE INVENTION

The present invention is generally related to treatments for ocular disorders and more specifically to the use of agents that lower IOP and/or treat or prevent glaucoma via down-regulation of PAI-1 expression or activity, thereby ameliorating PAI-1 mediated inhibition of the activity of tissue plasminogen activator (t-PA) and/or urokinase plasminogen activator (u-PA).

### BACKGROUND OF THE INVENTION

Primary open-angle glaucoma (POAG) is a common and devastating ophthalmic disease that causes progressive visual field loss if left untreated. A majority of glaucoma patients present with elevated intraocular pressure (IOP), and many current treatments are directed to lowering IOP elevation or maintaining normal IOP.

An increased level of plasminogen activator inhibitor-1 (PAI-1) appears to play a role in a variety of disease states, including cancer, obesity, and diabetes. Elevated levels of PAI-1 have been detected in the aqueous humor of glaucoma patients (Dan et al., Archives of Ophthalmology, Vol. 123:220-224, 2005). PAI-1 levels are increased by the cytokine TGFβ (Binder et al., News Physiol Science, Vol. 17:56-61, 2002), among other endogenous stimuli. PAI-1 inhibits the activity of both tissue plasminogen activator (t-PA) and urokinase plasminogen activator (u-PA). Both t-PA and u-PA catalyze the conversion of plasminogen into plasmin, a key intermediate in the fibrinolytic cascade (Wu et al., Current Drug Targets, Vol. 2:27-42, 2002). Plasmin is known to promote the conversion of certain pro-matrix metalloproteinases (MMPs) into their active, extracellular matrix (ECM)-degrading forms (He et al., PNAS, Vol. 86:2632-2636, 1989). PAI-1 also modulates the association of vitronectin, an ECM component, with cell surface integrins which act as adhesion receptors (Zhou et al., Nature Structural Biology, Vol. 10(7):541-544, 2003). Thus, PAI-1 has been linked to both decreased adhesion and increased detachment of cells in non-ocular tissues. Human ocular tissues also express t-PA and/or u-PA to varying degree; however the trabecular meshwork (TM) has been reported to predominantly express t-PA (Shuman et al., IOVS, Vol. 29:401-405, 1988; Tripathi et al., Exp Eye Research, Vol. 51:545-552, 1990). t-PA also appears to be the predominant form present in human aqueous humor (AH).

Drug therapies that have proven to be effective for the reduction of IOP and/or the treatment of POAG include both agents that decrease aqueous humor production and agents that increase the outflow facility. Such therapies are in general administered by one of two possible routes; topically (direct application to the eye) or orally. However, pharmaceutical ocular anti-hypertension approaches have exhibited various undesirable side effects. For example, miotics such as pilocarpine can cause blurring of vision, headaches, and other negative visual side effects. Systemically administered carbonic anhydrase inhibitors can also cause nausea, dyspepsia, fatigue, and metabolic acidosis. Certain prostaglandins cause hyperemia, ocular itching, and darkening of eyelashes and periorbital skin. Such negative side-effects may lead to decreased patient compliance or to termination of therapy such that vision continues to deteriorate. Additionally, there are individuals who simply do not respond well when treated with certain existing glaucoma therapies. There is, therefore, a need for other therapeutic agents for the treatment of ocular disorders such as glaucoma and ocular hypertension.

U.S. patent application Ser. No. 11/931,393, filed Dec. 15, 2006 and published as U.S. Patent Publication No. 2008/0107644, discloses potential use of agents which regulate the binding of PAI-1 to vitronectin as a means to prevent trabecular meshwork cell loss and, ultimately, lower intraocular pressure. The present invention is directed to the inhibition of PAI-1's effects on tissue plasminogen activator (t-PA) and/or urokinase plasminogen activator (u-PA).

### BRIEF SUMMARY OF THE INVENTION

Embodiments of the present invention are directed to the inhibition of PAI-1 expression or activity to treat ocular disease and/or lower IOP. One embodiment provides a method for treating glaucoma or elevated IOP in a patient comprising administering to the patient an effective amount of a composition comprising an agent that inhibits PAI-1 expression or prevents PAI-1 from inhibiting the activity of tissue plasminogen activator (t-PA) or urokinase plasminogen activator (u-PA).

Another embodiment of the present invention is a method of treating a PAI-1-associated ocular disorder comprising administering an effective amount of a composition comprising an agent that inhibits PAI-1 expression and/or PAI-1's effects on t-PA or u-PA activity.

In certain of these embodiments, the agent is tiplaxtinin (PAI-039), diaplasinin (PAI-749), ZK-4044, WAY-140312, HP-129, T-686, XR5967, XR334, XR330, XR5118, aleplasinin (PAZ-417), T-2639, S35225, SK216, SK116, 2-[2-methoxy-6-[[[3-(trifluoromethyl)-4-[4-[3-(trifluoromethyl)phenyl]-1-piperazinyl]phenyl]amino]methyl]phenoxy]-5-nitrobenzoic acid (also referred to herein as “Compound 39”; Ye et al., Bioorganic & Medicinal Chemistry Letters, Vol. 14(3):761-765, 2004) and combinations thereof. Other embodiments may use agents such as SB202190, U0126, SP600125, bisindolylmaleimide I, rottlerin, SB431542 and SIS3. Statin agents such as lovastatin, simvastatin, pravastatin, fluvastatin, atorvastatin may be used as agents in yet other embodiments. PAI-1 antibodies and peptidomimetics may also be used in certain embodiments. Combinations of such agents are also contemplated.

Yet another embodiment is a method of manufacturing a compound to be used as a treatment for glaucoma or elevated IOP comprising providing a candidate substance suspected of inhibiting PAI-1 expression or activity, selecting the compound by assessing the ability of the candidate substance to decrease the amount of PAI-1 in its active conformation in the trabecular meshwork of a subject suffering from glaucoma or elevated PAI-1, and manufacturing the selected compound.

In certain embodiments, compositions of the invention further comprise a compound selected from the group consisting of ophthalmologically acceptable preservatives, surfactants, viscosity enhancers, penetration enhancers, gelling agents, hydrophobic bases, vehicles, buffers, sodium chloride, water, and combinations thereof.

In yet other embodiments, a compound selected from the group consisting of β-blockers, prostaglandin analogs, carbonic anhydrase inhibitors, α₂ agonists, miotics, neuroprotectants, rho kinase inhibitors, and combinations thereof may be administered either as part of the composition or as a separate administration.

The foregoing brief summary broadly describes the features and technical advantages of certain embodiments of the present invention. Additional features and technical advantages will be described in the detailed description of the invention that follows. Novel features which are believed to be characteristic of the invention will be better understood from the detailed description of the invention when considered in connection with any accompanying figures. However, figures provided herein are intended to help illustrate the invention or assist with developing an understanding of the invention, and are not intended to be definitions of the invention's scope.

### BRIEF DESCRIPTION OF THE DRAWINGS

A more complete understanding of the present invention and the advantages thereof may be acquired by referring to the following description, taken in conjunction with the accompanying drawings.

FIG. 1 is a graph of experimental results showing the concentration-dependent effect of TGFβ2 (24 h) on levels of PAI-1 in human trabecular meshwork (GTM-3) cell supernatants. Data are expressed as mean and SEM, n=3. *p<0.05 versus corresponding vehicle group by one-way ANOVA, followed by the Dunnett test.

FIG. 2 is a graph of experimental results showing PAI-1 levels in GTM-3 cell supernatants with or without treatment with TGFβ2 (5 ng/mL) for various time periods. Data are expressed as mean and SEM, n=3. *p<0.05 versus corresponding vehicle time point group, by Student's t-test.

FIG. 3 is a graph showing the effect of TGFβ2 on total and active PAI-1 content in supernatants of treated GTM-3 cell cultures. Effect of TNFα and Dexamethasone are included for comparison. Data are mean and SEM after 24 h exposure to test agents; a value of “0” indicates levels of expression below the detection limit of the assay.

FIG. 4 shows two bar graphs summarizing the effect of PAI-1 inhibition on active PAI-1 in GTM-3 cell cultures.

FIG. 5 a graph showing the effects of a PAI-1 synthesis inhibitor (T-2639) on the TGFβ2-mediated increase of total PAI-1 protein levels in supernatants of treated GTM-3 cell cultures.

FIG. 6 shows graphs of the effect of TGFβ2 (5 ng/mL) in the presence or absence of the Type I TGFβ receptor inhibitor SB431542. Upper panel: Effect of SB431542 (10 μM) in various HTM cell lines. Lower panel: Dose-dependent effect of SB431542 on GTM-3 cells. Data are mean and SEM after 24 h exposure to test agents. (* denotes p<0.001 or ** denotes p<0.05 vs. the respective TGFβ2-treated control groups by One-way ANOVA then Bonferroni's test).

FIG. 7 shows graphs of the effect of TGFβ2 (5 ng/mL) in the presence or absence of the Smad3 inhibitor SIS3 (Jinnin et al., Molecular Pharmacology, Vol. 69:597-607, 2006). Upper panel: Effect of SIS3 (10 μM) in various HTM cell lines. Lower panel: Dose-dependent effect of SIS3 on GTM-3 cells. Data are mean and SEM after 24 h exposure to test agents. (* denotes p<0.001 vs. the respective TGFβ2-treated control groups by One-way ANOVA then Bonferroni's test).

FIG. 8 shows graphs of the effect of various intracellular signaling pathway enzyme inhibitors on TGFβ2-stimulated GTM-3 (Upper panel) and SGTM2697 (Lower panel) cells. Inhibitors used: SB202190 (p38 MAPK inhibitor), U0126 (MEK1/2 inhibitor), SP600125 (JNK inhibitor), Bisindolylmaleimide I (“Bis I”; PKCα, β, δ, ζ inhibitor), and Rottlerin (PKCδ inhibitor). Data are mean and SEM after 24 h exposure to test agents. (* denotes p<0.001 vs. TGFβ2-treated control group by One-way ANOVA then Bonferroni's test); and

FIG. 9 shows graphs of the effect of statins on TGFβ2-stimulated GTM-3 cells. Upper panel: Effect of various statins (10 μM). Lower panel: Dose-dependent effect of atorvastatin. Data are mean and SEM after 24 h exposure to test agents. (* denotes p<0.001 or ** denotes p<0.01 vs. TGFβ2-treated control group by One-way ANOVA then Bonferroni's test).

FIG. 10 is a series of graphs depicting the effect of compounds (tiplaxtinin, diaplasinin, and “Compound 39”) in a surrogate assay of extracellular matrix clearance. The tested compounds elicited demonstrable increases over basal (no treatment) activity in supernatant aliquots from each of six different HTM cell lines.

FIG. 11 presents two graphs of experimental results showing the effect of two compounds (tiplaxtinin and diaplasinin), which prevent the ability of PAI-1 to inhibit t-PA and u-PA activity, on Ad.TGFβ2-induced increase in intraocular pressure in Balb/cJ mice. IOP reduction was achieved by both pre- and post-dosing of PAI-1 inhibitors, with respect to Adv.TGFβ2-injection.

FIG. 12 presents two graphs of experimental results showing the effect of these same two inhibitors of PAI-1 (tiplaxtinin and diaplasinin) on Adv.PAI-1 induced increase in intraocular pressure in Balb/cJ mice.

### DETAILED DESCRIPTION OF THE INVENTION

Certain embodiments of the present invention are methods for targeting the effects of PAI-1 in ocular disorders such as glaucoma by interfering with PAI-1 activity relative to t-PA and u-PA and/or PAI-1 expression as shown in the following scheme,

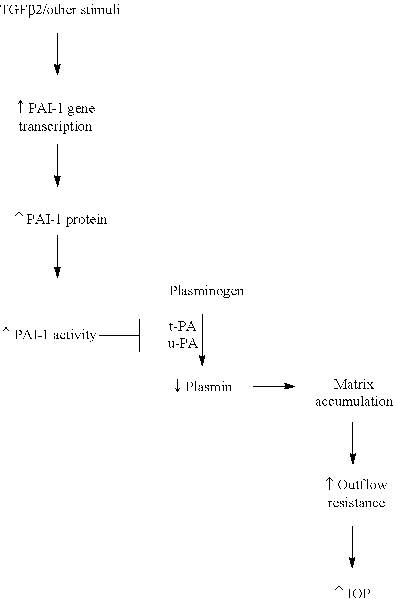

where TGFβ2 (or other stimuli) promotes PAI-1 gene transcription, followed by an increase in PAI-1 protein expression and increased levels of active PAI-1. Active PAI-1 inhibits conversion of plasminogen into plasmin by t-PA and/or u-PA. The subsequent decrease in plasmin levels reduces fibrinolytic capacity and increases extracellular matrix (ECM) accumulation. ECM accumulation increases outflow resistance and, ultimately, increases IOP. Embodiments of the present invention recognize that inhibition of PAI-1 expression and/or interfering with PAI-1 activity relative to t-PA and/or u-PA is a useful glaucoma therapy.

Various compounds that inhibit PAI-1 expression or activity are known in the art. U.S. patent application Ser. No. 11/611,312 (Fleenor et al., filed Dec. 15, 2006 and published as U.S. Patent Publication No. 2008/0107644) and U.S. Pat. No. 7,351,407 (Fleenor et al, issued Apr. 1, 2008) disclose compounds that may be useful as compounds that inhibit PAI-1 expression or activity, and are hereby incorporated by reference in their entirety.

The PAI-1 inhibitors of the present invention include, but are not limited to PAI-039 (tiplaxtinin) (Crandall et al., Arterioscler Thrombosis Vascular Biology Journal, Vol. 26(10):2209-2215, 2006); PAI-749 (diaplasinin) (Gardell et al., Molecular Pharmacology, Vol. 72(4):897-906, 2007); ZK4044 (Liang et al., Thrombosis Research, Vol. 115(4):341-350, 2005); WAY-140312 (Crandall et al., Journal Thrombosis Haemostasis, Vol. 2(8):1422-1428, 2004); HP-129 (fendosal) (Gils et al., Thrombosis Haemostasis, Vol. 88(1):137-143, 2002); T-686 (Murakami et al., Japanese Journal of Pharmacology, Vol. 75(3):291-294, 1997); PAZ-417 (aleplasinin) (Zhao et al., Cell Research, Vol. 18:803-804, 2008); T-2639 (Miyazaki et al., Biorganic & Medicinal Chemistry Letters, Vol. 18:6419-6422, 2008); S-35225 (Rupin et al., Thrombosis Research, Vol. 122:265-270, 2008; SK-216 & SK-116 (Mutoh et al., Carcinogenesis, Vol. 29(4):824-829, 2008); and 2-[2-methoxy-6-[[[3-(trifluoromethyl)-4-[4-[3-(trifluoromethyl)phenyl]-1-piperazinyl]phenyl]amino]methyl]phenoxy]-5-nitrobenzoic acid (“Compound 39”; (Ye et al., Bioorganic & Medicinal Chemistry Letters, Vol. 14(3):761-765, 2004).

Other small molecules such as piperazine and menthol derivatives (Ye et al., Bioorganic & Medicinal Chemistry Letters, Vol. 14(3):761-765, 2004; Ye et al., Biorganic & Medicinal Chemistry Letters, Vol. 13(19):3361-3365, 2003), PAI-1 antibodies (Verbeke et al., Journal of Thrombosis and Haemostasis, Vol. 2(2):289-297, 2004; van Giezen et al., Thrombosis and Haemostasis, Vol. 77(5):964-969, 1997; and Abrahamsson et al., Thrombosis and Haemostasis, Vol. 75(1):118-126, 1996), and protein agents such as paionin-4 (Mathiasen et al., Molecular Pharmacology, Vol. 74(3):641-653, 2008) may also be used as compounds that inhibit PAI-1 expression or activity in certain embodiments of the present invention.

Other embodiments may use agents such as SB202190, HP-129, U0126, SP600125, bisindolylmaleimide I, rottlerin, SB431542 and SIS3. Statin agents such as lovastatin, simvastatin, pravastatin, fluvastatin, atorvastatin may be used as agents in yet other embodiments. Preferred compounds that inhibit PAI-1 expression or activity are tiplaxtinin, diaplasinin, Compound 39 and T-2639.

The compounds that inhibit PAI-1 expression or activity of the present invention can be incorporated into various types of ophthalmic formulations for delivery. The compounds may be delivered directly to the eye (for example: topical ocular drops or ointments; slow release devices such as pharmaceutical drug delivery sponges implanted in the cul-de-sac or implanted adjacent to the sclera or within the eye; periocular, conjunctival, sub-tenons, intracameral, intravitreal, or intracanalicular injections) or systemically (for example: orally, intravenous, subcutaneous or intramuscular injections; parenteral, dermal or nasal delivery) using techniques well known by those of ordinary skill in the art. It is further contemplated that the PAI-1 expression or activity inhibitors of the invention may be formulated in intraocular inserts or implantable devices.

The PAI-1 expression or activity inhibitors disclosed herein are preferably incorporated into topical ophthalmic formulations for delivery to the eye. The compounds may be combined with ophthalmologically acceptable preservatives, surfactants, viscosity enhancers, penetration enhancers, buffers, sodium chloride, and water to form an aqueous, sterile ophthalmic suspension or solution. Ophthalmic solution formulations may be prepared by dissolving a compound in a physiologically acceptable isotonic aqueous buffer. Further, the ophthalmic solution may include an ophthalmologically acceptable surfactant to assist in dissolving the compound. Furthermore, the ophthalmic solution may contain an agent to increase viscosity such as hydroxymethylcellulose, hydroxyethylcellulose, hydroxypropylmethylcellulose, methylcellulose, polyvinylpyrrolidone, or the like, to improve the retention of the formulation in the conjunctival sac. Gelling agents can also be used, including, but not limited to, gellan and xanthan gum. In order to prepare sterile ophthalmic ointment formulations, the active ingredient is combined with a preservative in an appropriate vehicle such as mineral oil, liquid lanolin, or white petrolatum. Sterile ophthalmic gel formulations may be prepared by suspending the compound in a hydrophilic base prepared from the combination of, for example, carbopol-974, or the like, according to the published formulations for analogous ophthalmic preparations; preservatives and tonicity agents can be incorporated.

PAI-1 expression or activity inhibitors are preferably formulated as topical ophthalmic suspensions or solutions, with a pH of about 4 to 8. The compounds are contained in the topical suspensions or solutions in amounts sufficient to lower IOP in patients experiencing elevated IOP and/or maintaining normal IOP levels in glaucoma patients. Such amounts are referred to herein as “an amount effective to control IOP,” or more simply “an effective amount.” The compounds will normally be contained in these formulations in an amount 0.01 to 5 percent by weight/volume (“w/v %”), but preferably in an amount of 0.25 to 2 w/v %. Thus, for topical presentation 1 to 2 drops of these formulations would be delivered to the surface of the eye 1 to 4 times per day, according to the discretion of a skilled clinician.

The PAI-1 expression or activity inhibitors may also be used in combination with other elevated IOP or glaucoma treatment agents, such as, but not limited to, rho kinase inhibitors, β-blockers, prostaglandin analogs, carbonic anhydrase inhibitors, α₂ agonists, miotics, serotonergic agonists and neuroprotectants.

As used herein, “PAI-1 expression or activity inhibitor” encompasses such inhibitors as well as their pharmaceutically-acceptable salts. A pharmaceutically acceptable salt of a PAI-1 expression or activity inhibitor is a salt that retains PAI-1 expression or activity inhibitory activity and is acceptable by the human body. Salts may be acid or base salts since agents herein may have amino or carboxy substituents. A salt may be formed with an acid such as acetic acid, benzoic acid, cinnamic acid, citric acid, ethanesulfonic acid, fumaric acid, glycolic acid, hydrobromic acid, hydrochloric acid, maleic acid, malonic acid, mandelic acid, methanesulfonic acid, nitric acid, oxalic acid, phosphoric acid, propionic acid, pyruvic acid, salicylic acid, succinic acid, sulfuric acid, tartaric acid, p-toluenesulfonic acid, trifluoroacetic acid, and the like. A salt may be formed with a base such as a primary, secondary, or tertiary amine, aluminum, ammonium, calcium, copper, iron, lithium, magnesium, manganese, potassium, sodium, zinc, and the like.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

PAI-1 expression or activity inhibitors can be selected using binding assays or functional assays that can also be used to determine their biological activity. Such assays can be developed by those of skill in the art using previously described methods. Other useful assays for selecting PAI-1 expression or activity inhibitors are presented in Examples 2-5.

The ability of certain PAI-1 expression or activity inhibitors to safely lower IOP may be evaluated by means of in vivo assays. In one such assay using Cynomolgus monkeys, IOP is determined with an Alcon pneumatonometer after light corneal anesthesia with 0.1% proparacaine. (Sharif et al., Journal Ocular Pharmacology & Therapeutics, Vol. 17(4):305-317, 2001; May et al., Journal of Pharmacology & Experimental Therapeutics, Vol. 306(1):301-309, 2003). Eyes are rinsed with one or two drops of saline after each measurement. After a baseline IOP measurement, test compound is instilled in one or two 30 μL aliquots to the selected eye. Subsequent IOP measurements are taken at 1, 3, and 6 hours. Right eyes of all animals undergo laser trabeculoplasty to induce ocular hypertension. All left eyes are normal and thus have normal IOP.

In another assay using New Zealand Albino rabbits, IOP is determined with a Mentor Classic 30 pneumatonometer after light corneal anesthesia with 0.1% proparacaine. Eyes are rinsed with one or two drops of saline after each measurement. After a baseline IOP measurement, test compound is instilled in one 30 μL aliquot to one or both eye of each animal or compound to one eye and vehicle to the contralateral eye. Subsequent IOP measurements are taken at 0.5, 1, 2, 3, 4, and 5 hours.

### Example 2

Human TM cells were isolated from post-mortem human donor tissue, characterized, and cultured as previously described. Generation and characterization of the transformed (GTM-3) cell line was as previously described by Pang et al. (Current Eye Research, Vol. 13(1):51-63, 1994). 24-well plates of TM cell cultures were serum-deprived for 24 h followed by an additional 24 h (or as indicated) incubation with TGFβ2 in a serum-free medium. Aliquots of supernatants from the treated cultures were quantified for secreted PAI-1 content by means of human PAI-1 ELISA kit (Imubind; American Diagnostica Inc., Greenwich, Conn.). The ELISA detects both latent and active PAI-1, as well as PAI-1 complexes, with a minimum detectable limit of 50 pg/mL.

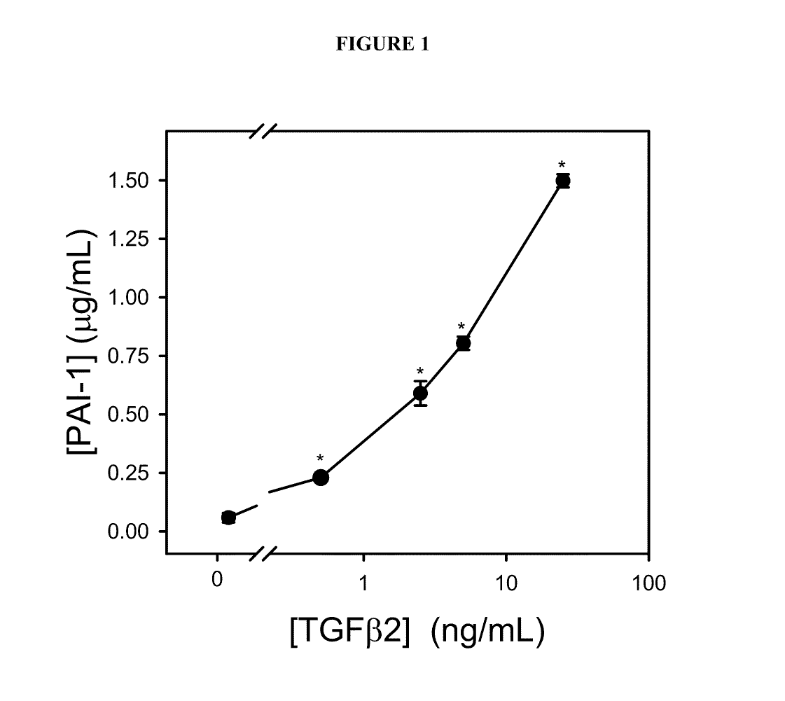

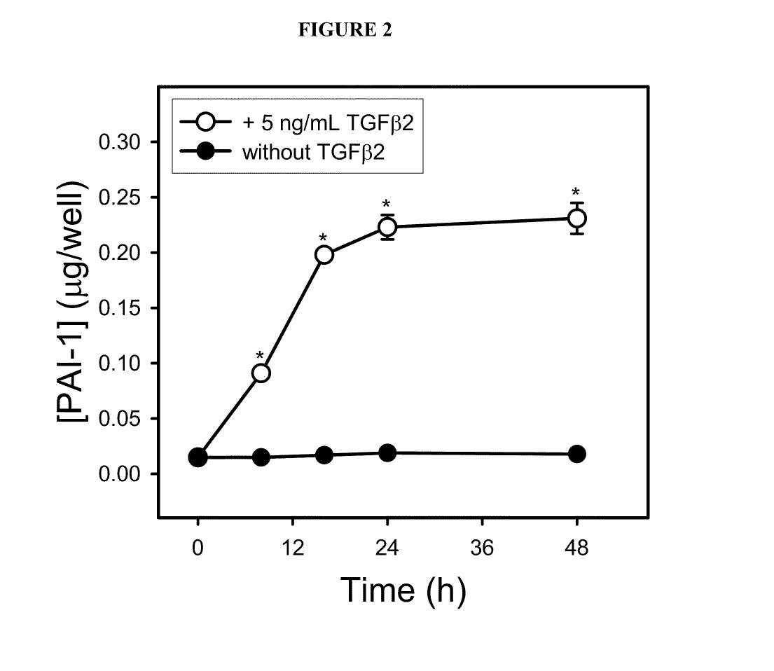

FIG. 1 is a graph showing that TGFβ2 increases the PAI-1 content in trabecular meshwork cell cultures (GTM-3). PAI-1 mediated effects may contribute to the previously observed TGFβ2-mediated accumulation of extracellular matrix materials in various tissues, including TM tissues. FIG. 2 demonstrates that such TGFβ2-mediated PAI-1 increases are persistent in cell cultures treated with TGFβ2. Accordingly, TGFβ2-treatment appears to result in both concentration-dependent and time-dependent accumulation of PAI-1 in TM cell supernatants. PAI-1 levels increase gradually in response to TGFβ2, reaching a constant level at approximately 24 h post-treatment.

### Example 3

Transforming growth factor-beta (TGFβ3) regulates the production of a wide variety of gene and protein products and, thus, multiple cellular processes. Studies have shown that ex vivo treatment of human trabecular meshwork (HTM) cells with the TGFβ2 isoform leads to changes in expression of plasminogen activator inhibitor-1 (PAI-1), an important mediator that likely contributes to ocular extracellular matrix (ECM) accumulation. A disproportionate accretion of ECM in the TM region may impart greater resistance to aqueous humor (AH) outflow and, consequently, increased intraocular pressure, such as seen in primary open angle glaucoma. Additionally, levels of both TGFβ2 and PAI-1 are greater in AH collected from human POAG eyes as compared to non-glaucomatous eyes. Furthermore, ex vivo human anterior segments respond with decreases in outflow facility when perfused with TGFβ2.

In these studies, human TM cells were isolated, characterized, and cultured as previously described (Pang et al., Current Eye Research, Vol. 13(1):51-63, 1994). For these assays, plated cells were serum-deprived for 24 h followed by additional 24 h incubation with test agents in a serum-free medium. Aliquots of supernatants from treated cultures were then removed for quantification of total PAI-1 content by means of a human PAI-1 ELISA kit (Imubind; American Diagnostica Inc., Greenwich, Conn.). The ELISA detects both latent and active PAI-1, as well as PAI-1 complexes, with a minimum detectable limit of 50 pg/mL. Active PAI-1 content in the cell supernatants was evaluated with an ELISA kit (Molecular Innovations, Southfield, Mich.) that quantifies binding of active PAI-1 to urokinase. Latent and complexed PAI-1 does not bind urokinase and thus is not detected by the assay. The expected detection limit of the assay is approximately 0.045 U/mL (where 1 Unit equals approximately 1.34 ng active PAI-1).

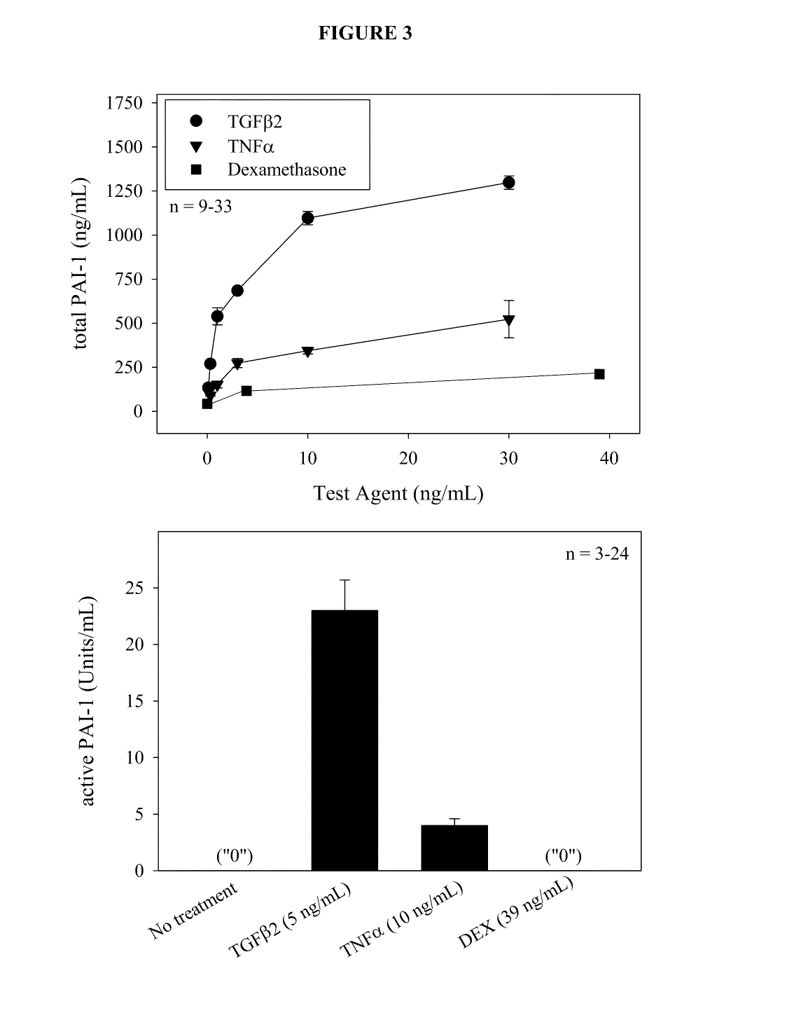

FIGS. 3-9 present the results of in vitro experiments conducted using the above protocols. The average basal PAI-1 secretion by GTM-3 cells in the studies was 33.9±1.5 ng/mL/24 h (n=233). TGFβ2 increased PAI-1 content of GTM-3 cell supernatants in a time and dose-dependent manner. A 24 h treatment with 5 ng/mL TGFβ2 elevated PAI-1 levels by 12.02±0.03 fold.

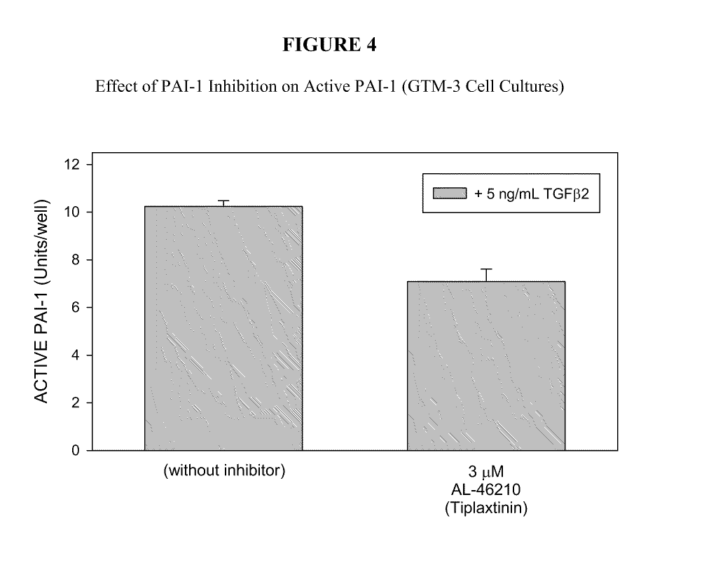

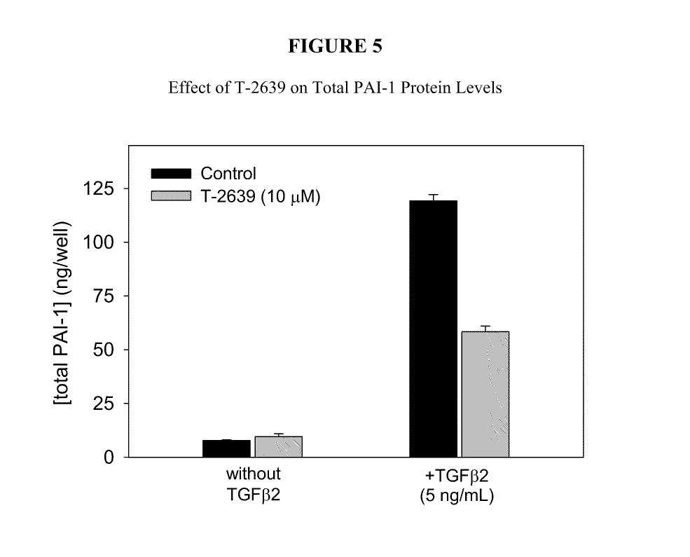

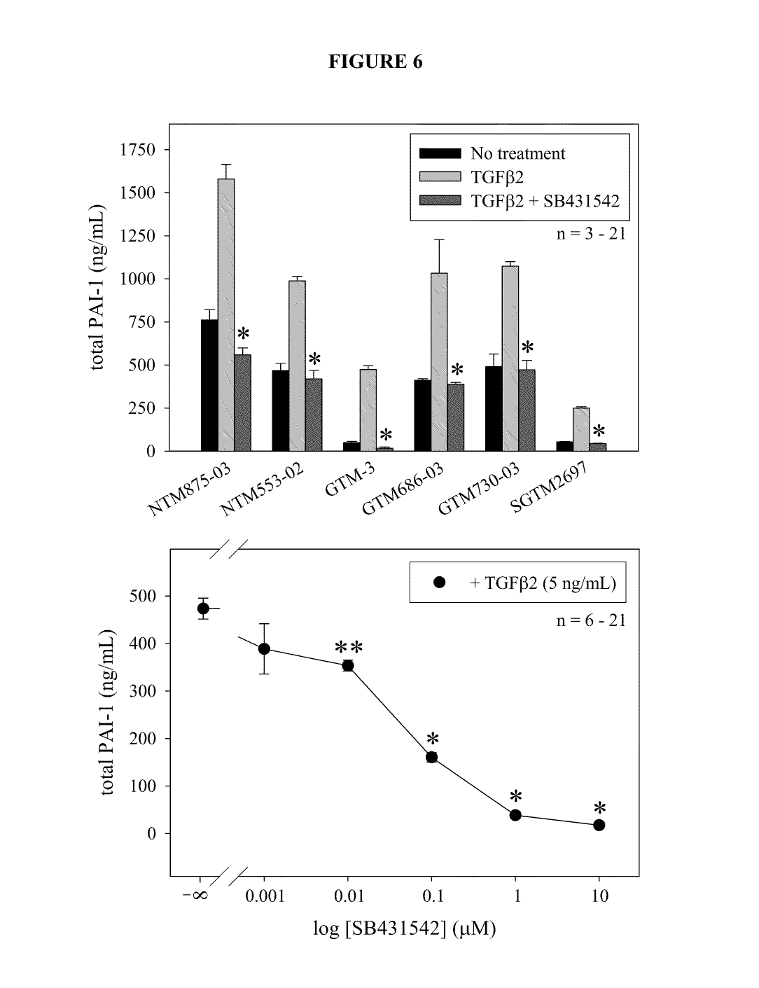

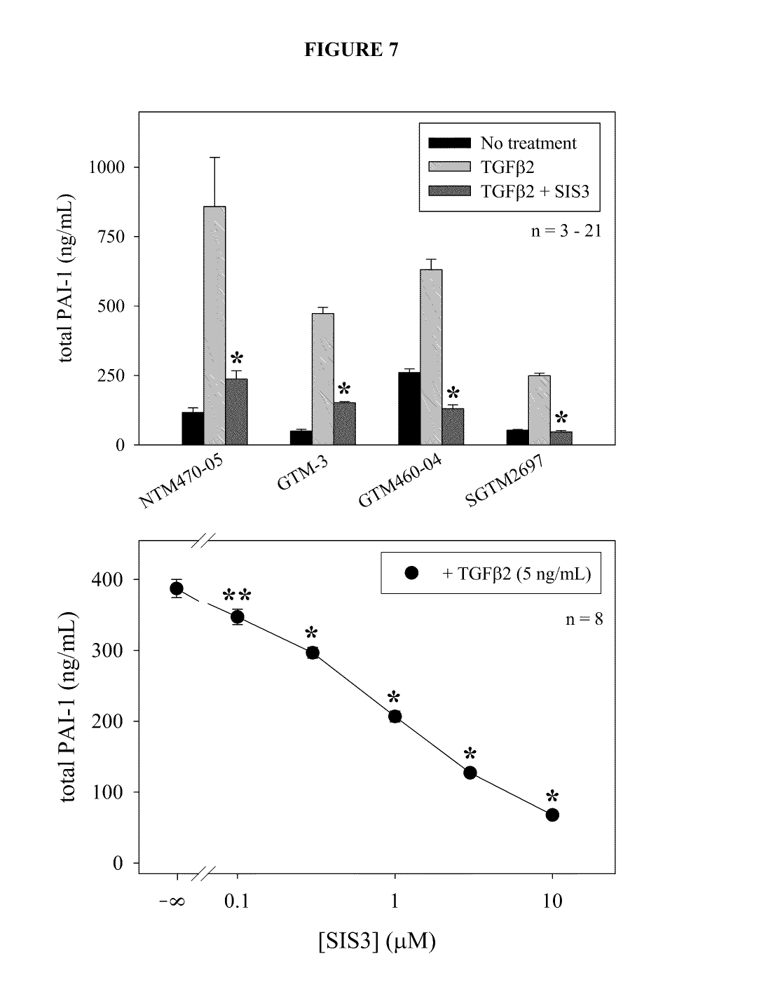

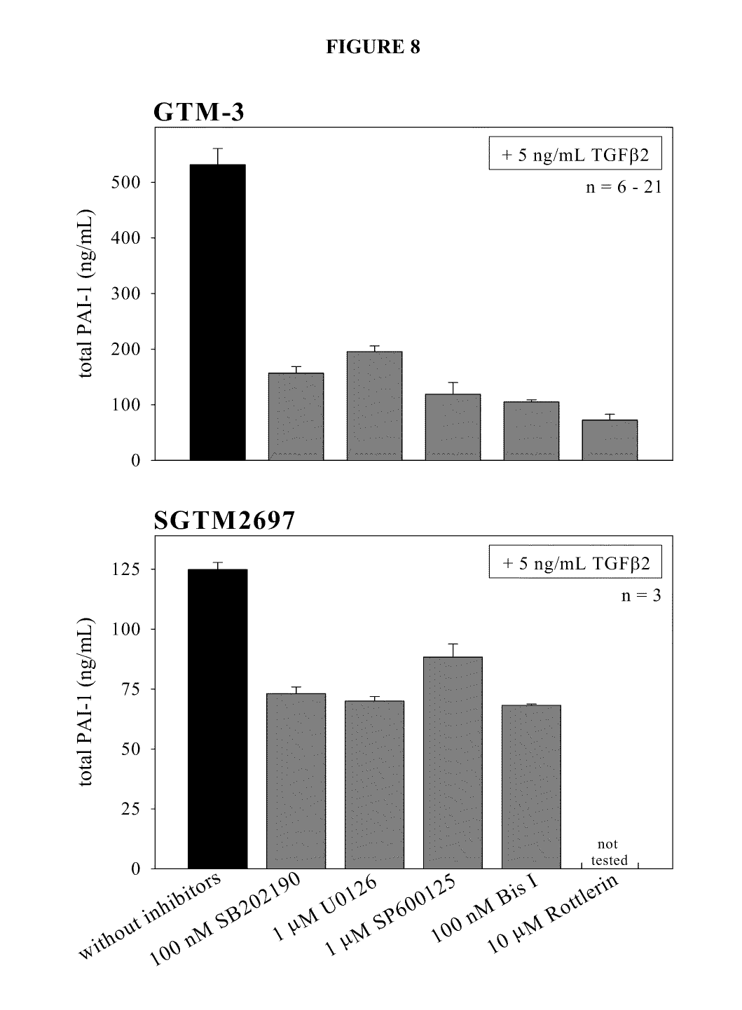

HTM cell PAI-1 total protein levels are upregulated in vitro by factors (TGFβ2, TNFα, dexamethasone) linked to increased intraocular pressure. Active PAI-1 levels are also increased by TGFβ2 (FIG. 3). FIG. 4 shows that tiplaxtinin reduces active PAI-1 levels in GTM-3 cultures treated with TGFβ2. TGFβ2-stimulated PAI-1 levels were significantly (p<0.05) down-regulated by inhibitors of both canonical (Smad-mediated) and non-canonical (Smad-independent) signal transduction pathways. FIG. 5 is a graph showing the effects of a PAI-1 synthesis inhibitor (T-2639) on the TGFβ2-mediated increase of total PAI-1 protein levels in supernatants of treated GTM-3 cell cultures. Inhibitors of TGFβ2-mediated canonical (Smad) signaling pathways (SB431542 and SIS3) block the in vitro expression of total PAI-1 in human trabecular meshwork (HTM) cell cultures (FIGS. 6 and 7). Inhibitors of TGFβ2-mediated non-canonical (Smad-independent) signaling pathways (SB202190, U0126, SP600125, bisindolylmaleimide I, and rottlerin) also prevent in vitro expression of total PAI-1 in HTM cell cultures. Such signaling pathways identified to date include p38 MAPK, MEK1/2, JNK, and PKC\tilde{δ} (FIG. 8).

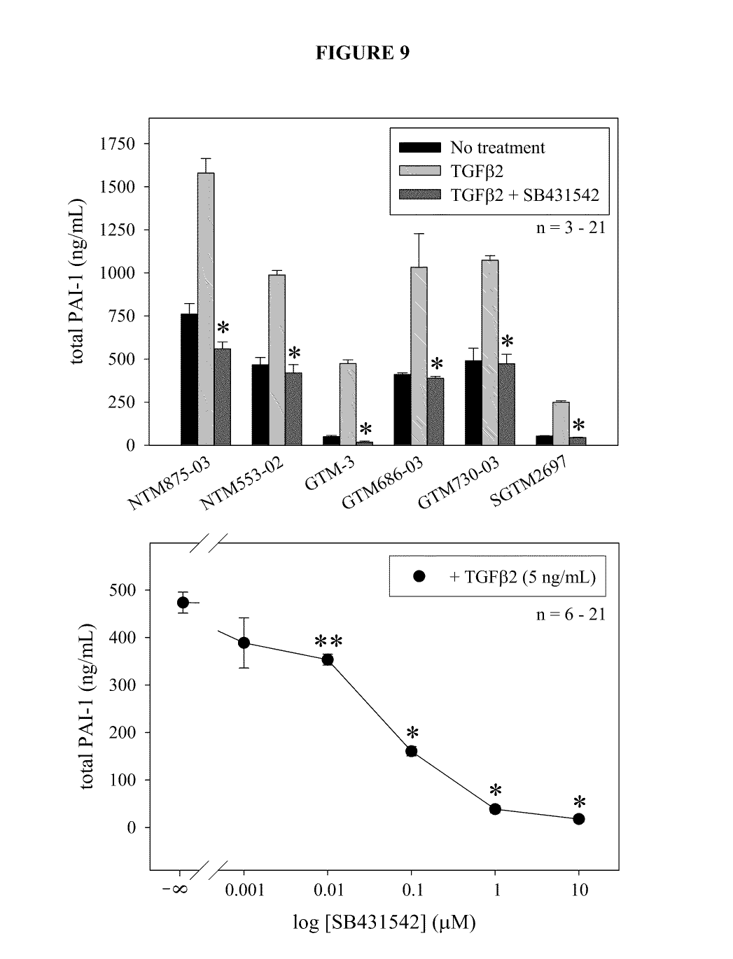

Treatment with statin agents also decreases in vitro expression of total PAI-1 in HTM cell cultures. (FIG. 9). Overall response varied from complete inhibition by agents such as SB431542 (TGFβ Type 1 receptor inhibitor; 1 μM) and rottlerin (PKCδ inhibitor; 10 μM) to partial inhibition by SB202190 (p38 MAPK inhibitor; 100 nM), SP600125 (c-Jun N-terminal kinase inhibitor; 1 μM), and various statin agents.

### Example 4

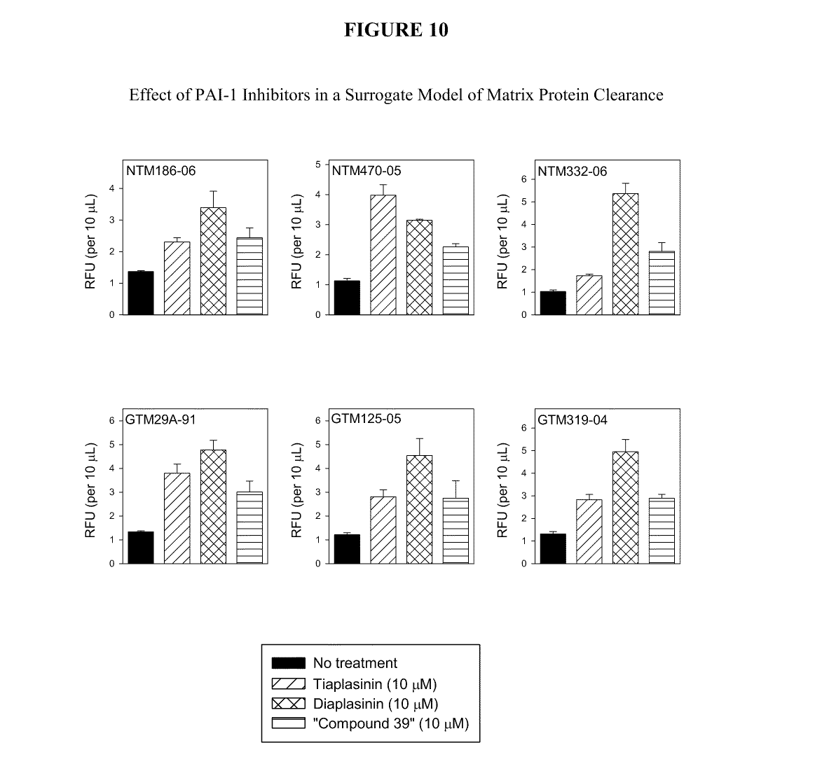

A study was conducted to evaluate the effect of the compounds of the present invention on extracellular matrix clearance. Human TM cells are treated for 24 h in the presence or absence of tiplaxtinin, diaplasinin, and Compound 39. Cell supernatant aliquots are then incubated for 2 h with IRDye 800RS-labeled casein (Li-Cor Biosciences), followed by detection of accumulated fluorescent degradation products with an Odyssey Infrared Imaging System (Li-Cor Biosciences). FIG. 10 shows that tiplaxtinin, diaplasinin, and Compound 39 elicited demonstrable increases over basal (no treatment) activity in supernatant aliquots from each of six different HTM cell lines. Accordingly, treatment with these compounds enhances the degradation of matrix protein by trabecular meshwork cells.

### Example 5

To evaluate the in vivo effects of the compounds of the present invention, a mouse model was used. One eye each of BALB/cJ mice was injected intravitreally with either Ad5.CMV.hPAI-1 or Ad.CMV.hTGFβ2^{226/228}. Un-injected contralateral eyes served as controls. IOP was measured in conscious animals at selected time points via rebound tonometer (TonoLab®). Test agents were administered via daily topical dosing (bid) during the time frames indicated on the graphs.

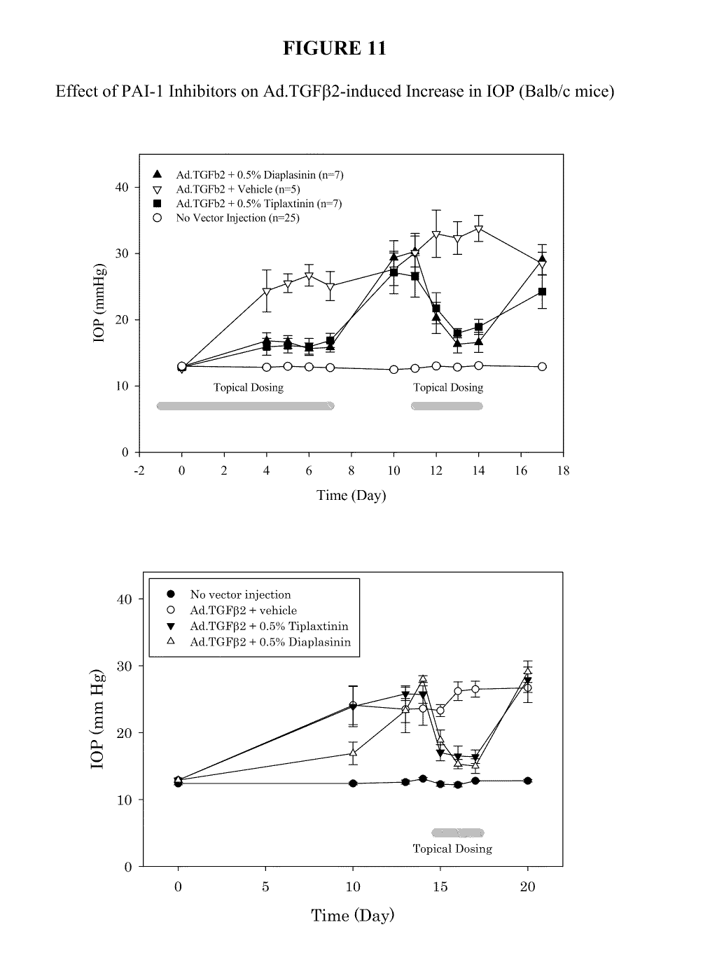

FIG. 11 presents two graphs of experimental results showing the effect of compounds (tiplaxtinin and diaplasinin) that inhibit the inhibitory activity of PAI-1 on t-PA and u-PA. The compounds almost completely reverse Ad.TGFβ2-induced increase in intraocular pressure in Balb/C mice. IOP reduction was achieved by both pre- and post-dosing of PAI-1 inhibitors, with respect to Adv.TGFβ2-injection.

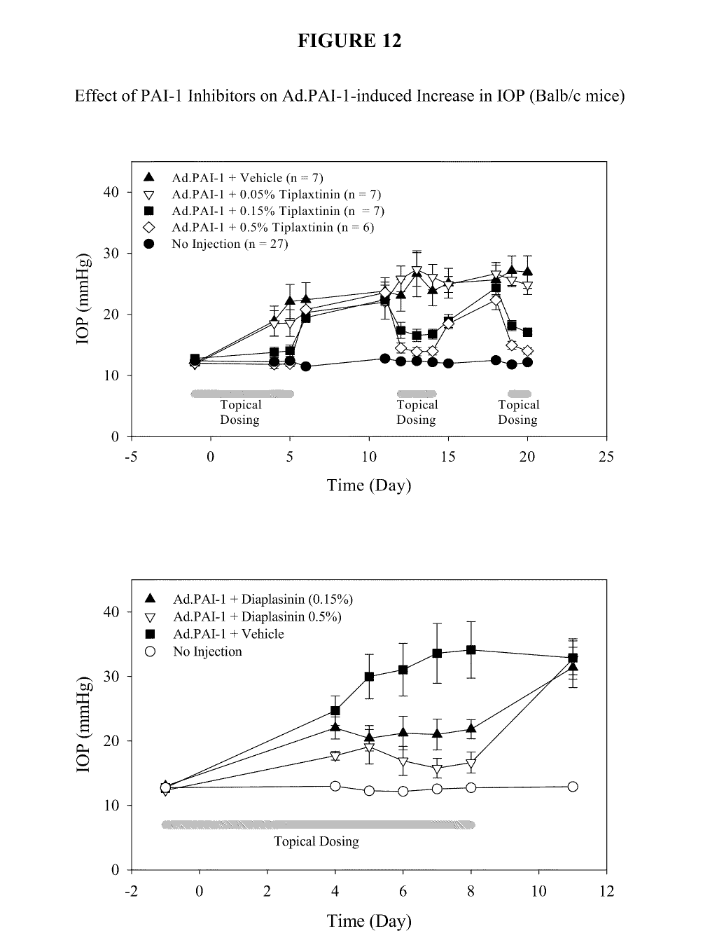

FIG. 12 presents two graphs of experimental results showing the effect of two compounds (tiplaxtinin and diaplasinin) that prevent the inhibitory activity of PAI-1 on t-PA and u-PA. Both agents reduced the Ad.PAI-1 induced increase in intraocular pressure in Balb/cJ mice.

### Example 6

| Ingredients | Concentration (w/v %) |
|---|---|
| Tiplaxtinin | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 7

| Ingredients | Concentration (w/v %) |
|---|---|
| Diaplasinin | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

The present invention and its embodiments have been described in detail. However, the scope of the present invention is not intended to be limited to the particular embodiments of any process, manufacture, composition of matter, compounds, means, methods, and/or steps described in the specification. Various modifications, substitutions, and variations can be made to the disclosed material without departing from the spirit and/or essential characteristics of the present invention. Accordingly, one of ordinary skill in the art will readily appreciate from the disclosure that later modifications, substitutions, and/or variations performing substantially the same function or achieving substantially the same result as embodiments described herein may be utilized according to such related embodiments of the present invention. Thus, the following claims are intended to encompass within their scope modifications, substitutions, and variations to processes, manufactures, compositions of matter, compounds, means, methods, and/or steps disclosed herein.

### REFERENCES

The following references are hereby incorporated by reference in their entirety:
ABRAHAMSSON et al., “Anti-thrombotic Effect of a PAI-1 Inhibitor in Rats Given Endotoxin”, Thrombosis and Haemostasis, Vol. 75(1):118-26, 1996
BINDER et al., “Plasminogen Activator Inhibitor 1: Physiological and Pathophysiological Roles”, News Physiol Science, Vol. 17:56-61, 2002
CRANDALL et al., “Characterization and Comparative Evaluation of a Structurally Unique PAI-1 Inhibitor Exhibiting Oral in-vivo Efficacy”, Journal Thrombosis Haemostasis, Vol. 2(8):1422-8, 2004
CRANDALL et al., “Modulation of Adipose Tissue Development by Pharmacological Inhibition of PAI-1”, Arterioscler Thrombosis Vascular Biology Journal, Vol. 26(10):2209-2215, 2006
DAN et al., “Plasminogen Activator Inhibitor-1 in the Aqueous Humor of Patients With and Without Glaucoma”, Archives of Ophthalmology, Vol. 123:220-224, 2005
GARDELL et al., “Neutralization of Plasminogen Activator Inhibitor I (PAI-1) by the Synthetic Antagonist PAI-749 via a Dual Mechanism of Action,” Molecular Pharmacology, Vol. 72(4):897-906, 2007
GILS et al., “Characterization and Comparative Evaluation of a Novel PAI-1 Inhibitor”, Thrombosis Haemostasis, Vol. 88(1):137-143, 2002
HE et al., “Tissue Cooperation in a Proteolytic Cascade Activating Human Interstitial Collagenase”, PNAS, Vol. 86:2632-2636, 1989
JINNIN et al., “Characterization of SIS3, a Novel Specific Inhibitor of Smad3, and its Effect on Transforming Growth Factor-B1-Induced Extracellular Matrix Expression”, Molecular Pharmacology, Vol. 69:597-607, 2006
LIANG et al, “Characterization of a Small Molecule PAI-1 Inhibitor, ZK4044”, Thrombosis Research, Vol. 115(4):341-350, 2005
MATHIASEN et al., “A Peptide Accelerating the Conversion of Plasminogen Activator Inhibitor-1 to an Inactive Latent State”, Molecular Pharmacology, Vol. 74(3):641-653, 2008
MAY et al., “Evaluation of the Ocular Hypotensive Response of Serotonin 5-HT1A and 5-HT2 Receptor Ligands in Conscious Ocular Hypertensive Cynomolgus Monkeys”, Journal of Pharmacology & Experimental Therapeutics, Vol. 306(1):301-309, 2003
MIYAZAKI et al., “Design, Synthesis, and Evaluation of Orally Active Inhibitors of Plasminogen Activator Inhibitor-1 (PAI-1) Production”, Bioorganic & Medicinal Chemistry Letters, Vol. 18:6419-6422, 2008
MURAKAMI et al., “Protective Effect of T-686, an Inhibitor of Plasminogen Activator Inhibitor-1 Production, Against the Lethal Effect of Lipopolysaccharide in Mice,” Japanese Journal of Pharmacology, Vol. 75(3):291-294, 1997
MUTOH et al., “Plasminogen Activator Inhibitor-1 (Pai-1) Blockers Suppress Intestinal Polyp Formation in Min Mice”, Carcinogenesis, Vol. 29(4):824-829, 2008
PANG et al., “Preliminary Characterization of a Transformed Cell Strain Derived from Human Trabecular Meshwork”, Current Eye Research, Vol. 13(1):51-63, 1994
RUPIN et al., “S35225 is a Direct Inhibitor of Plasminogen Activator Inhibitor Type-1 Activity in the Blood”, Thrombosis Research, Vol. 122:265-270, 2008
SHARIF et al., “Levobetaxolol (Betaxon™) and Other B-Adrenergic Antagonists: Preclinical Pharmacology, IOP-Lowering Activity and Sites of Action in Human Eyes”, Journal of Ocular Pharmacology and Therapeutics, Vol. 17(4):305-317, 2001
SHUMAN et al., “Tissue Plasminogen Activator in Cultured Human Trabecular Meshwork Cells”, IOVS, Vol. 29:401-405, 1988
TRIPATHI et al., “Aqueous Humor in Glaucomatous Eyes Contains an Increased Level of TGF-beta 2”, Exp Eye Research, Vol. 59:723-727, 1994
TRIPATHI et al., “Localization of Urokinase-type Plasminogen Activator in Human Eyes: An Immunocytochemical Study”, Exp Eye Research, Vol. 51:545-552, 1990
van GIEZEN et al., “The Fab-fragment of a PAI-1 Inhibiting Antibody Reduces Thrombus Size and Restores Blood Flow in a Rat Model of Arterial Thrombosis”, Thrombosis and Haemostasis, Vol. 77(5):964-969, 1997
VERBEKE et al., “Cloning and Paratope Analysis of an Antibody Fragment, a Rational Approach for the Design of a PAI-1 inhibitor”, Journal of Thrombosis and Haemostasis, Vol. 2(2):289-297, 2004
WU et al., “Inhibition of PAI-1: A New Anti-thrombotic Approach”, Current Drug Targets, Vol. 2:27-42, 2002
YE et al., “Synthesis and Biological Evaluation of Menthol-based Derivatives as Inhibitors of Plasminogen Activator Inhibitor-1 (PAI-1)”, Bioorganic Medicinal Chemistry Letters, Vol. 13(19):3361-3365, 2003
YE et al., “Synthesis and Biological Evaluation of piperazine-Based Derivatives as Inhibitors of Plasminogen Activator Inhibitor-1 (PAI-1)”, Bioorganic & Medicinal Chemistry Letters, Vol. 14(3):761-765, 2004
ZHAO et al., “Evoking Plasmin for β-amyloid Clearance”, Cell Research, Vol. 18:803-804, 2008
ZHOU et al., “How Vitronectin Binds PAI-1 to Modulate Fibrinolysis and Cell Migration”, Nature Structural Biology, Vol. 10(7):541-544, 2003
U.S. Pat. No. 7,351,407 (Fleenor et al, issued Apr. 1, 2008)
U.S. Patent Publication No. 2008/0107644 Fleenor et al., Published May 8, 2008

What is claimed is:

1. A method for treating glaucoma or elevated IOP in a patient comprising:
administering to the patient an effective amount of a composition comprising an agent that inhibits PAI-1 expression or activity.

2. A method according to claim 1 wherein said agent is selected from the group consisting of:
tiplaxtinin, diaplasinin, aleplasinin, fendosal, ZK4044, WAY-140312, T-686, T-2639, S-35225, SK-216, SK-116, SB202190, U0126, HP-129, SP600125, XR5967, XR334, XR330, XR5118, Compound 39, bisindolylmaleimide I, rottlerin, SB431542, SIS3, statins, lovastatin, simvastatin, pravastatin, fluvastatin, atorvastatin, PAI-1 antibodies, PAI-1 protein inhibitors, paionin-4, and combinations thereof.

3. A method according to claim 2 wherein said agent is selected from the group consisting of:
aleplasinin, fendosal, T-2639, S-35225, SK-216, SK-116, SB202190, U0126, SP600125, Compound 39, bisindolylmaleimide I, rottlerin, SB431542, SIS3, statins, lovastatin, simvastatin, pravastatin, fluvastatin, atorvastatin, PAI-1 antibodies, PAI-1 protein inhibitors, paionin-4, and combinations thereof.

4. A method according to claim 3 wherein said agent is selected from the group consisting of:
tiplaxtinin, diaplasinin, bisindolylmaleimide I, T-2639, Compound 39, and combinations thereof.

5. A method according to claim 1 wherein said inhibition interferes with PAI-1 inhibition of tissue plasminogen activator (t-PA) or urokinase plasminogen activator (u-PA) activity.

6. A method according to claim 1 wherein said composition further comprises a compound selected from the group consisting of:
ophthalmologically acceptable preservatives, surfactants, viscosity enhancers, penetration enhancers, gelling agents, hydrophobic bases, vehicles, buffers, sodium chloride, water, and combinations thereof.

7. A method according to claim 1, further comprising administering, either as part of said composition or as a separate administration, a compound selected from the group consisting of:
β-blockers, prostaglandin analogs, carbonic anhydrase inhibitors, α₂ agonists, miotics, neuroprotectants, rho kinase inhibitors, and combinations thereof.

8. A method according to claim 1 wherein said composition comprises from about 0.01 percent weight/volume to about 5 percent weight/volume of said agent.

9. A method according to claim 1 wherein said composition comprises from about 0.25 percent weight/volume to about 2 percent weight/volume of said agent.

10. A method of treating a PAI-1-associated ocular disorder in a subject in need thereof, comprising:
administering to the patient an effective amount of a composition comprising an agent that inhibits PAI-1 activity or expression.

11. A method according to claim 10 wherein the disorder is ocular hypertension or glaucoma.

12. A method according to claim 10 wherein said agent is selected from the group consisting of:
tiplaxtinin, diaplasinin, aleplasinin, fendosal, ZK4044, WAY-140312, T-686, T-2639, S-35225, SK-216, SK-116, SB202190, U0126, HP-129, SP600125, XR5967, XR334, XR330, XR5118, Compound 39, bisindolylmaleimide I, rottlerin, SB431542, SIS3, statins, lovastatin, simvastatin, pravastatin, fluvastatin, atorvastatin, PAI-1 antibodies, PAI-1 protein inhibitors, paionin-4, and combinations thereof.

13. A method according to claim 12 wherein said agent is selected from the group consisting of:
aleplasinin, fendosal, T-2639, S-35225, SK-216, SK-116, SB202190, U0126, SP600125, Compound 39, bisindolylmaleimide I, rottlerin, SB431542, SIS3, statins, lovastatin, simvastatin, pravastatin, fluvastatin, atorvastatin, PAI-1 antibodies, PAI-1 protein inhibitors, paionin-4, and combinations thereof.

14. A method according to claim 13 wherein said agent is selected from the group consisting of:
tiplaxtinin, diaplasinin, bisindolylmaleimide I, T-2639, Compound 39, and combinations thereof.

15. A method according to claim 10 wherein said inhibition prevents PAI-1 inhibition of tissue plasminogen activator (t-PA) or urokinase plasminogen activator (u-PA) activity.

16. A method according to claim 10 wherein said composition further comprises a compound selected from the group consisting of:
ophthalmologically acceptable preservatives, surfactants, viscosity enhancers, penetration enhancers, gelling agents, hydrophobic bases, vehicles, buffers, sodium chloride, water, and combinations thereof.

17. A method according to claim 10, further comprising administering, either as part of said composition or as a separate administration, a compound selected from the group consisting of:
β-blockers, prostaglandin analogs, carbonic anhydrase inhibitors, α₂ agonists, miotics, neuroprotectants, rho kinase inhibitors, and combinations thereof.

18. A method according to claim 10 wherein said composition comprises from about 0.01 percent weight/volume to about 5 percent weight/volume of said agent.

19. A method according to claim 10 wherein said composition comprises from about 0.25 percent weight/volume to about 2 percent weight/volume of said agent.
